(19) United States
(12) Patent Application Publication
(10) Pub. No.: US 2013/0338656 A1
(43) Pub. Date: Dec. 19, 2013

(54) TREATMENT SYSTEM AND ACTUATION METHOD FOR TREATMENT SYSTEM

(71) Applicant: OLYMPUS MEDICAL SYSTEMS CORP., Tokyo (JP)

(72) Inventors: Takashi IRISAWA, Tokyo (JP); Kazue TANAKA, Sagamihara-shi (JP); Yoshitaka HONDA, Tokyo (JP); Sadayoshi TAKAMI, Tokyo (JP)

(21) Appl. No.: 13/921,514

(22) Filed: Jun. 19, 2013

Related U.S. Application Data
(63) Continuation of application No. PCT/JP2012/079371, filed on Nov. 13, 2012.
(60) Provisional application No. 61/569,325, filed on Dec. 12, 2011.

(51) Int. Cl. A61B 18/12

(52) U.S. Cl. CPC A61B 18/12
USPC 606/27; 607/98

(57) ABSTRACT

A treatment system includes a high-frequency power source, a heat generation power source, a grasping member having an electric conductor which applies high-frequency power energy to a living tissue and a heating element which applies thermal energy, a memory where a predetermined impedance value or a predetermined rate of increase in impedance that is a threshold value, at which application of the high-frequency power energy is ended, is stored in advance, a first control section which controls the high-frequency power source based on the threshold value to end application of the high-frequency power energy, and a second control section which performs constant temperature control on the heat generation power source such that the heating element reaches a setting temperature that is set to be higher than a temperature of the heating element calculated from a resistance value of the heating element when application of the high-frequency power energy ends.

### CROSS REFERENCE TO RELATED APPLICATION

This application is a continuation application of PCT/JP2012/079371 filed on Nov. 13, 2012 and claims benefit of U.S. Provisional Patent Application No. 61/569,325 filed in the U.S.A. on Dec. 12, 2011, the entire contents of which are incorporated herein by this reference.

### BACKGROUND OF THE INVENTION

1. Field of the Invention

Embodiments of the present invention relate to a treatment system including one pair of grasping members which apply high-frequency power energy and thermal energy to a grasped living tissue in turn and an actuation method for the treatment system.

2. Description of the Related Art

U.S. Patent Application Publication No. 2009/076506 discloses a treatment system including one pair of grasping members which apply high-frequency power energy and thermal energy to a grasped living tissue, a high-frequency power source which outputs high-frequency power for applying high-frequency power energy, a power source for heat generation which outputs power for heat generation for applying thermal energy, and a control section which controls the high-frequency power source and the power source for heat generation for switching between application of high-frequency power energy and application of thermal energy.

U.S. Patent Application Publication No. 2009/0248002 discloses a treatment system which first applies high-frequency power energy to a living tissue and then starts application of thermal energy.

U.S. Pat. No. 5,558,671 discloses an idea of calculating a target impedance Ztarget, at which coagulation is complete, from a minimum impedance Zmin which is detected by monitoring an impedance Z for high-frequency power and ending application of high-frequency power energy when the impedance Z reaches the target impedance Ztarget, at the time of treating a living tissue with high-frequency power energy.

U.S. Patent Application Publication No. 2007/173803 discloses an idea of calculating a threshold value impedance indicating an end of treatment by adding, as an offset impedance, a minimum impedance Zmin which is detected after treatment starts to a predetermined impedance (ending impedance) which is set in advance and corresponds to a tissue.

### SUMMARY OF THE INVENTION

A treatment system according to an embodiment includes a high-frequency power source which outputs high-frequency power, a power source for heat generation which outputs power for heat generation, a grasping member having an electric conductor which applies the high-frequency power as high-frequency power energy to a living tissue and a heating element which applies the power for heat generation as thermal energy to the living tissue, a memory where a predetermined impedance value or a predetermined rate of increase in impedance that is a threshold value, at which application of the high-frequency power energy is ended, is stored in advance, a first control section which controls the high-frequency power source on the basis of the threshold value so as to end application of the high-frequency power energy, and a second control section which performs constant temperature control on the power source for heat generation such that the heating element reaches a setting temperature that is set to be higher than a temperature of the heating element calculated from a resistance value of the heating element when application of the high-frequency power energy ends.

An actuation method for a treatment system according to another embodiment includes a step in which a treatment condition is set for the treatment system including a first control section which controls the high-frequency power source for applying high-frequency power as high-frequency power energy to an electric conductor and a second control section which controls the power source for heat generation for applying power for heat generation as thermal energy to a heating element, a step of performing, by the first control section, constant power control on the high-frequency power source on the basis of the treatment condition to start application of the high-frequency energy, a step of acquiring, by the first control section, a predetermined impedance value or a predetermined rate of increase in impedance that is a threshold value, at which application of the high-frequency power energy is ended, from a memory, a step of controlling, by the first control section, the high-frequency power source to automatically end application of the high-frequency energy on the basis of the threshold value, a step of applying, by the second control section, the thermal energy under constant temperature control on the power source for heat generation such that the heating element reaches a setting temperature that is set to be higher than a temperature of the heating element when application of the high-frequency energy is ended which is calculated from resistance of the heating element, and a step of controlling, by the second control section, the power source for heat generation on the basis of the treatment condition so as to end application of the thermal energy.

### BRIEF DESCRIPTION OF THE DRAWINGS

FIG. 1 is an external view of a treatment system according to a first embodiment;

FIG. 2 is a three-dimensional cross-sectional view for explaining a structure of a jaw of the treatment system according to the first embodiment;

FIG. 3 is a configuration diagram of the treatment system according to the first embodiment;

FIG. 4 is an external view of a treatment system according to a modification of the first embodiment;

FIG. 5 is a flowchart for explaining a flow of processing of the treatment system according to the first embodiment;

FIG. 6 is a graph showing change in impedance in high-frequency power application mode in the treatment system according to the first embodiment;

FIG. 7 is a graph showing change in temperature and change in power for heat generation in thermal energy application mode in the treatment system according to the first embodiment;

FIG. 8 is a flowchart for explaining a flow of processing of a treatment system according to a second embodiment;

FIG. 9 is a graph showing change in impedance in high-frequency power application mode in a treatment system according to a third embodiment;

FIG. 10 is a graph showing change in impedance and change in temperature in high-frequency power application mode in a treatment system according to a fourth embodiment; and

FIG. 11 is a graph showing change in impedance and change in temperature in high-frequency power application mode in a treatment system according to a fifth embodiment.

### DETAILED DESCRIPTION OF THE PREFERRED EMBODIMENTS

### First Embodiment

### Configuration of Treatment System

A treatment system 10 according to a first embodiment will first be described.

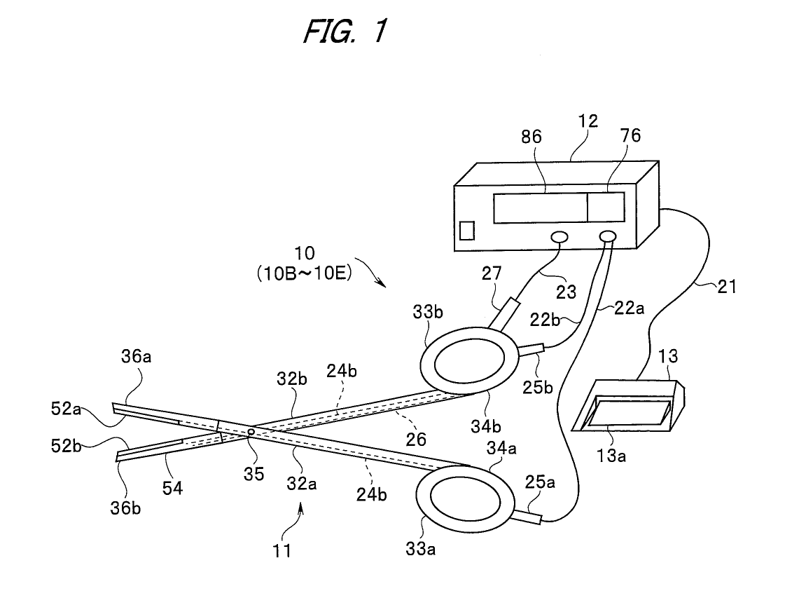

As shown in FIG. 1, the treatment system 10 includes a treatment instrument 11, a power supply portion 12, and a foot switch 13. The treatment system 10 switches between high-frequency power energy and thermal energy and applies, using the power supply portion 12, the energy to a living tissue grasped by jaws 36a and 36b which are one pair of grasping members of the treatment instrument 11. Note that high-frequency power and power for heat generation may hereinafter be abbreviated as “HF” and “TH,” respectively. For example, high-frequency power energy is referred to as HF energy.

The treatment instrument 11 is connected to the power supply portion 12 by HF lines 22a and 22b and a TH line 23. Note that although the HF lines 22a and 22b, the TH line 23, and the like each have two pieces of wiring, the two pieces of wiring are expressed as one line. The foot switch 13 is connected to the power supply portion 12 by a switch line 21.

The treatment instrument 11 has one pair of scissors constituent members 32a and 32b, one pair of handle portions 34a and 34b, and the one pair of jaws 36a and 36b. The handle portions 34a and 34b are provided at proximal end portions of the scissors constituent members 32a and 32b. The handle portions 34a and 34b are operated while being held in a hand by a surgeon. The jaws 36a and 36b are provided at distal end portions of the scissors constituent members 32a and 32b to grasp a living tissue to be treated.

The scissors constituent members 32a and 32b are placed one on the other so as to substantially intersect each other between distal ends and proximal ends of the scissors constituent members 32a and 32b. A fulcrum pin 35 which pivotably couples the scissors constituent members 32a and 32b is provided at an intersection portion of the scissors constituent members 32a and 32b.

The handle portions 34a and 34b are provided with rings 33a and 33b on which a surgeon puts his or her fingers. When the surgeon puts a thumb and a middle finger through the rings 33a and 33b, respectively, and opens and closes the rings 33a and 33b, the jaws 36a and 36b open and close in tandem with the motion.

Respective energy release elements which apply energy to a grasped living tissue are disposed at the jaws 36a and 36b. That is, an electrode 52a as the energy release element which has a grasping surface and is made of an electric conductor is disposed at the jaw 36a. An electrode 52b as the energy release element which has a grasping surface and is made of an electric conductor and a heater member 53 as a heating element are disposed at the jaw 36b. The heater member 53 is embedded in the jaw 36b while the heater member 53 is disposed on a reverse surface of the electrode 52b that is made of a high thermal conductor.

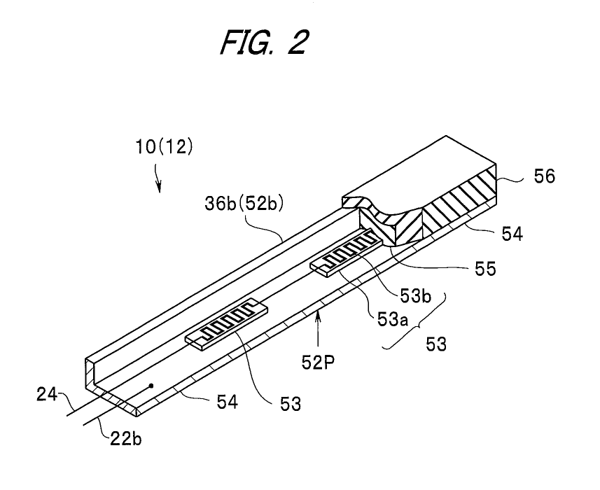

That is, as shown in FIG. 2, in the jaw 36b of the treatment instrument 11, the heater member 53 is joined to a surface reverse to a grasping surface 52P of a base 54 which is made of copper. The heater member 53 is covered with a sealing member 55 and a cover member 56. Note that FIG. 2 shows a portion of the jaw 36b and that three or more heater members 53 may be joined to each jaw 36b.

In the heater member 53, a thin-film resistor or a thick-film resistor is disposed as a heating pattern 53b on a substrate 53a of, e.g., alumina or aluminum nitride. The thin-film resistor is made of, e.g., an electrically conductive thin film which is formed by a thin film formation method, such as PVD (physical vapor deposition) or CVD (chemical vapor deposition), or foil of an electrically conductive metal, such as SUS. The thick-film resistance is formed by a thick film formation method, such as screen printing. The heating pattern 53b is formed of a refractory metal material, such as molybdenum, which exhibits a positive temperature coefficient of resistance indicating that electric resistance increases directly with temperature.

Note that the heater member 53 may also be disposed at the jaw 36a of the treatment instrument 11. That is, a heating element only needs to be disposed at at least one grasping member.

HF lines 24a and 24b for supplying HF to the electrodes 52a and 52b are disposed inside the scissors constituent members 32a and 32b, respectively. The HF lines 24a and 24b extend from the jaws 36a and 36b to the handle portions 34a and 34b, respectively. HF terminals 25a and 25b are provided at the rings 33a and 33b, respectively. The HF terminals 25a and 25b are connected to the HF lines 24a and 24b, respectively. For the reason, when HF is supplied to the electrodes 52a and 52b while a living tissue is grasped by the jaws 36a and 36b, HF is passed through the living tissue between the electrodes 52a and 52b. In other words, HF energy is applied to the living tissue.

A TH line 26 for supplying TH to the heater member 53 is disposed inside the scissors constituent member 32b. The TH line 26 extends from the jaw 36b to the handle portion 34b. A TH terminal 27 which is connected to the TH line 26 is provided at the ring 33b. For the reason, when TH is supplied to the heater member 53 through the TH line 26, the heater member 53 generates heat. That is, the TH is converted into thermal energy by the heater member 53, the thermal energy is transferred to the electrode 52b, and the thermal energy is applied to a living tissue in contact with the grasping surface of the electrode 52b.

As described above, when HF is passed between the electrodes 52a and 52b, the treatment instrument 11 applies HF energy to a living tissue grasped between the jaws 36a and 36b. When TH is passed through the heater member 53 in the treatment instrument 11, the TH is converted into the thermal energy. The treatment instrument 11 applies the thermal energy to the living tissue.

The foot switch 13 has a pedal 13a. While the pedal 13a is pressed, the power supply portion 12 outputs HF or TH on the basis of a setting state (a state in which an output value, output timing, and the like are controlled). When the press of the pedal 13a is canceled, the power supply portion 12 forcibly stops outputting the power.

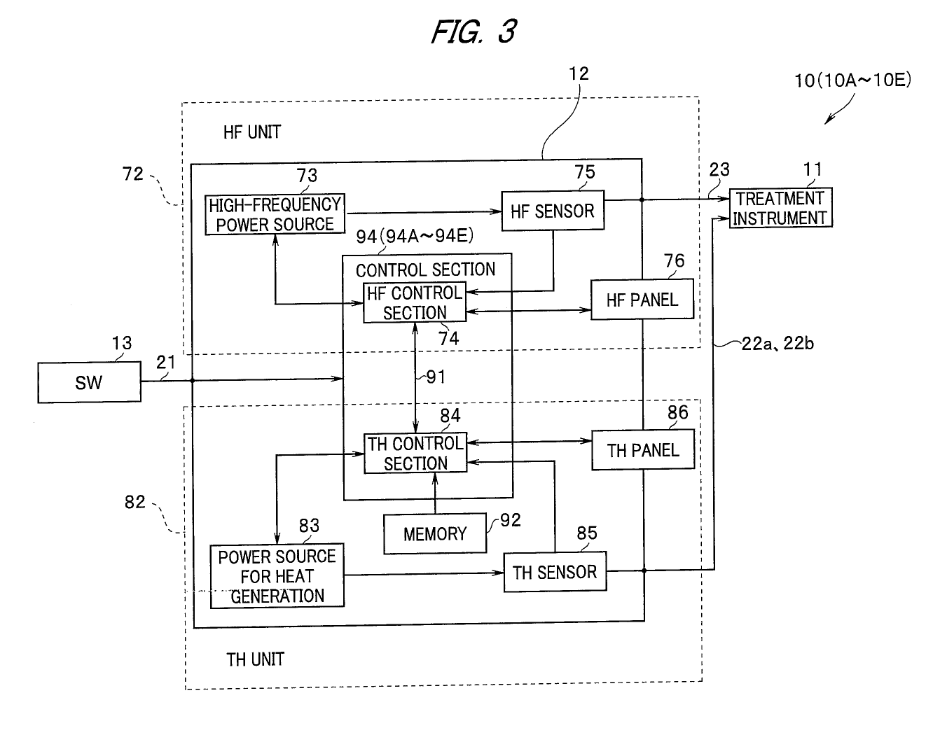

As shown in FIG. 3, the power supply portion 12 is composed of an HF unit 72 and a TH unit 82. The HF unit 72 has a high-frequency power source 73 which outputs HF, an HF control section 74 which is a first control section that controls the high-frequency power source 73 and is made up of a computing circuit, such as a CPU, an HF sensor 75 which is a high-frequency power measuring section which measures voltage and current of HF outputted by the high-frequency power source 73, and an operation panel 76.

The TH unit 82 has a power source 83 for heat generation which outputs TH, a TH control section 84 which is a second control section that controls the power source 83 for heat generation and is made up of a computing circuit, such as a CPU, a TH sensor 85 which is a heat generation power measuring section that measures voltage and current of TH outputted by the power source 83 for heat generation, an operation panel 86, and a memory 92 which is a storage section that is made up of, e.g., a semiconductor memory.

The HF control section 74 and the TH control section 84 are connected by a communication line 91 which can transmit a signal in both directions to constitute a control section 94. The control section 94 controls the high-frequency power source 73 and the power source 83 for heat generation. The operation panels 76 and 86 each have a setting function portion, with which a surgeon sets a treatment condition, and a display function of displaying a treatment status.

The HF sensor 75 is connected to the treatment instrument 11 via the HF lines 22a and 22b. The high-frequency power source 73 and the HF sensor 75 are connected to the HF control section 74. The HF control section 74 is further connected to the operation panel 76. The HF control section 74 calculates HF information, such as power and impedance, on the basis of information from the HF sensor 75, sends a control signal to the high-frequency power source 73, and sends information to be displayed to the operation panel 76. HF outputted by the high-frequency power source 73 that is controlled by the HF control section 74 is transmitted to the electrodes 52a and 52b of the treatment instrument 11.

The TH control section 84 calculates temperature of the heater member 53 as TH information on the basis of information from the TH sensor 85, in addition to power, a resistance value, and the like. That is, since the heating pattern of the heater member 53 is made of a material having a positive temperature coefficient of resistance, as already described, the TH control section 84 can calculate the temperature of the heater member 53 from a TH resistance value which is calculated from voltage and current of TH. The TH control section 84 sends a control signal to the power source 83 for heat generation on the basis of the TH information. TH outputted by the power source 83 for heat generation that is controlled by the TH control section 84 is transmitted to the heater member 53 of the treatment instrument 11.

Note that the HF control section 74 also sends a control signal to the TH control section 84 at the end of application of HF such that the TH control section 84 starts outputting TH.

As described above, the treatment instrument 11 has a function of a bipolar-type high-frequency treatment instrument and a function of a treatment instrument for heat generation.

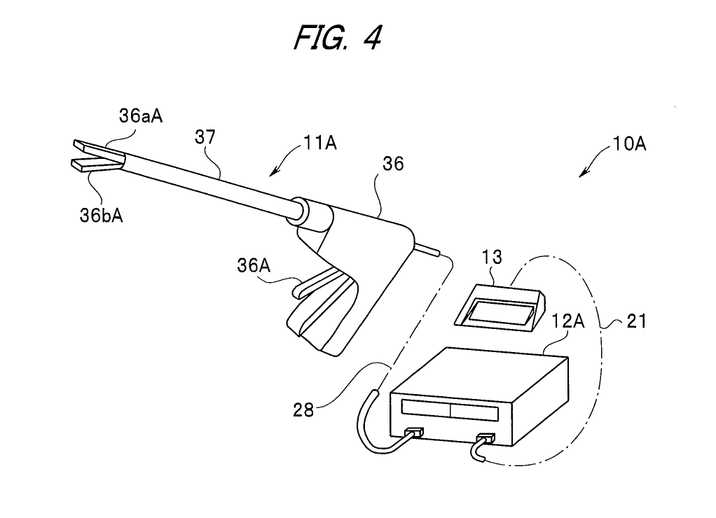

Note that the treatment instrument of the treatment system according to the embodiment may be a so-called linear-type treatment instrument. For example, a treatment system 10A according to a modification shown in FIG. 4 includes a linear-type treatment instrument 11A, a power supply portion 12A, and the foot switch 13.

The treatment instrument 11A has a handle 36, a shaft 37, and one pair of jaws 36aA and 36bA which are grasping members that grasp a living tissue. Structures of the jaws 36aA and 36bA are identical to the structures of the jaws 36a and 36b.

The handle 36 has a shape which is easy for a surgeon to grip, such as a substantially L-shape. The handle 36 has an open/close knob 36A. The open/close knob 36A is designed such that the jaws 36a and 36b grasp a living tissue when a surgeon presses and operates the open/close knob 36A. HV electrodes (not shown) and heater members (not shown) of the jaws 36aA and 36bA are connected to the power supply portion 12A via a piece 28 of wiring. That is, the piece 28 of wiring is made up of the HF lines 22a and 22b and the TH line 23. A basic configuration and a function of the power supply portion 12A are identical to the basic configuration and the function of the power supply portion 12.

That is, any of treatment instruments having various structures can be used as long as the treatment instrument can apply high-frequency power energy and thermal energy to a grasped living tissue.

### <Motion of Treatment System>

An actuation method for the treatment system 10 will be described.

The treatment system 10 first applies HF energy to a grasped living tissue and applies thermal energy after the application of HF energy ends. In other words, the control section 94 controls the high-frequency power source 73 and the power source 83 for heat generation to start applying thermal energy after application of high-frequency power energy ends.

That is, the treatment system 10 automatically switches from HF energy application mode to thermal energy application mode when a process of destroying cell membranes in the living tissue is completed through application of HF energy. In thermal energy application mode, moisture is removed by further raising temperature of the living tissue, and a process of joining the living tissue is performed through hydrogen bonding.

High-frequency power energy acts to release intracellular components including polymer compounds typified by protein by destroying cell membranes in the living tissue and make the intracellular components uniformed with extracellular components typified by collagen. High-frequency power energy also acts to raise the temperature of the living tissue.

Uniforming of the living tissue and the rise in temperature of the living tissue promote dehydration and joining of the living tissue through subsequent application of thermal energy. If high-frequency power energy is applied for too short a time period, undestroyed cell membranes remain, and the force with which the living tissue is joined through application of thermal energy is insufficient. On the other hand, if high-frequency power energy is applied for too long a time period, the living tissue may be excessively cauterized to cause local unevenness in burning or occurrence of arc discharge may generate a local overcurrent to cause damage to the tissue.

For the reason, when to end application of high-frequency power energy, i.e., when to switch from application of high-frequency power energy to application of thermal energy is important.

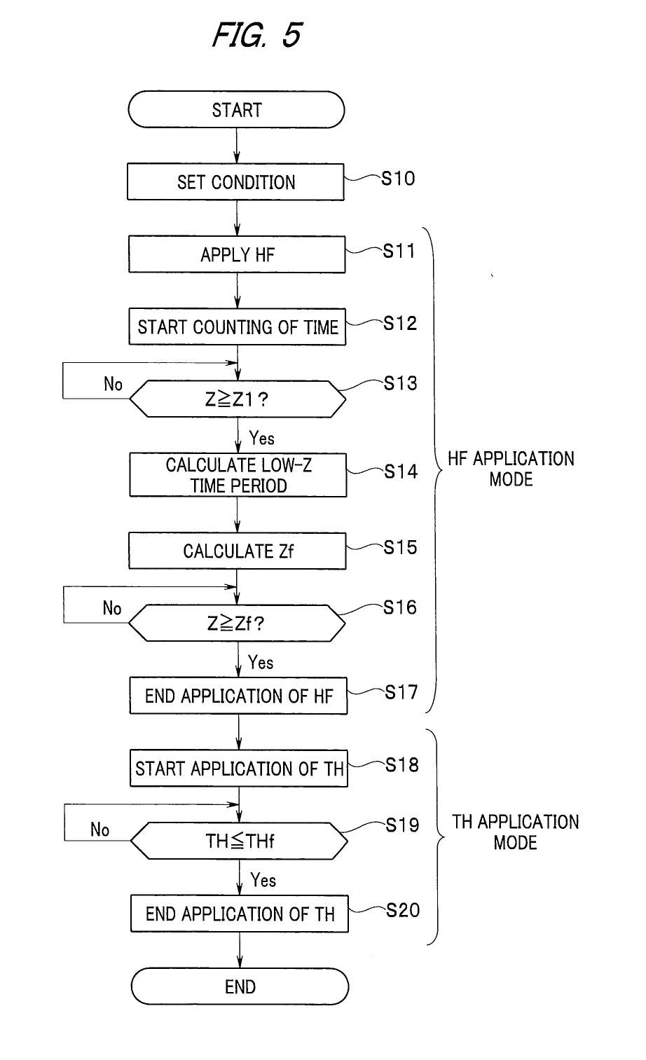

A motion of the treatment system 10 will be described below with reference to a flowchart shown in FIG. 5.

### <Step S10>

A surgeon inputs and sets treatment conditions to the control section 94 by using the operation panels 76 and 86. Examples of the treatment conditions include setting power Pset (W) for HF energy application mode, an impedance Z1 (W) which is a first predetermined value, a setting temperature Tset (° C.) for thermal energy application mode, and end power THE (W) for determining when to end thermal energy application mode. Note that the treatment conditions will be described in detail later.

### <Step S11>

The surgeon puts his or her fingers on the rings 33a and 33b of the handle portions 34a and 34b of the treatment instrument 11, operates the treatment instrument 11, and grasps a living tissue to be treated with the jaws 36a and 36b.

When the surgeon presses the pedal 13a of the foot switch 13 with a foot, application of HV energy to the living tissue between the electrodes 52a and 52b of the jaws 36a and 36b of the treatment instrument 11 starts. Note that the pedal 13a remains pressed during treatment. When the surgeon takes the foot off the pedal 13a, the power supply portion 12 forcibly stops outputting the energy.

HV outputted by the high-frequency power source 73 is controlled by constant power control to the predetermined setting power Pset (e.g., about 20 W to 150 W).

In HF energy application mode, Joule heat is generated to heat the living tissue itself. Dielectric breakdown, electric discharge, and the like arising from HF action destroy cell membranes in the living tissue. With the destruction of the cell membranes, released materials from the cell membranes are uniformed with extracellular components typified by collagen.

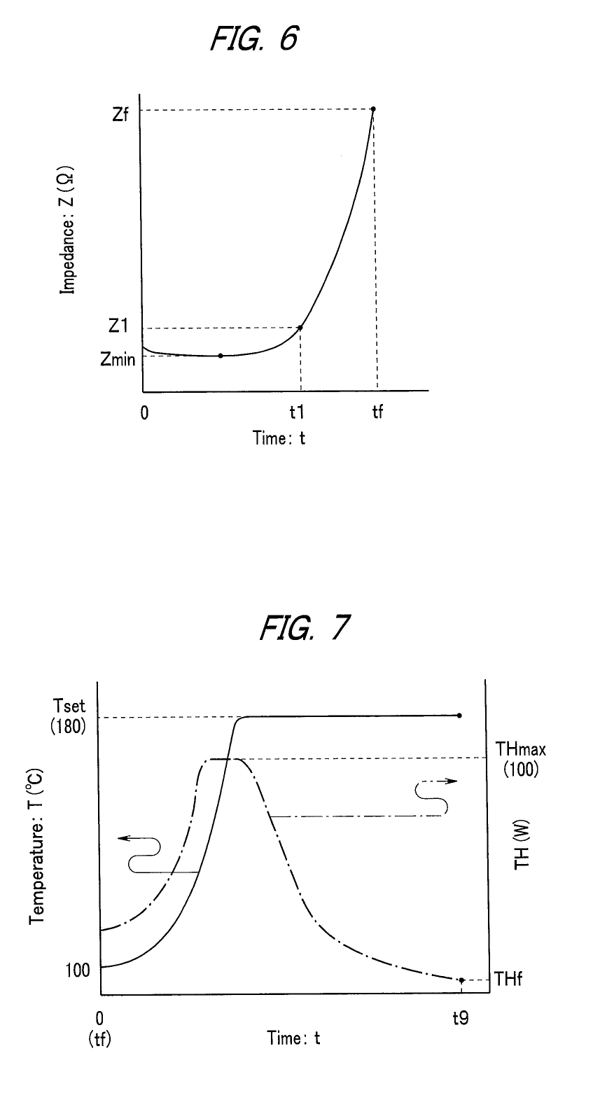

In HF energy application mode, an impedance Z for HF, i.e., the impedance Z in the grasped living tissue is calculated on the basis of HF information from the HF sensor 75. As shown in FIG. 6, the impedance Z is, for example, about 60Ω at the start of the HF energy application.

### <Step S12>

In the treatment system 10, when application of HF energy starts, the control section 94 starts measuring time together with the impedance Z for HF. The impedance Z is calculated from voltage and current of TH measured by the HF sensor 75 that is a high-frequency power measuring section.

### <Step S13>

When the HF energy application under constant power control (feedback control) starts, cell membranes in the grasped living tissue are destroyed, and substances in the cell membranes are released, which causes the impedance Z to decrease, as shown in FIG. 6. Since the release of substances from cell membranes and drying by HF energy proceed simultaneously, the impedance remains low for some time.

As many of cells in the grasped living tissue are destroyed, the amount of substances released from cell membranes decreases, and drying of the living tissue advances, which causes the impedance Z for HF to start rising. That is, the impedance Z rises after reaching a minimum impedance Zmin.

The control section 94 waits (NO) until the impedance Z becomes not less than the impedance Z1 that is the first predetermined value. The impedance Z1 is set according to the type of a tissue to be treated in step S10. The impedance Z1 is, for example, 20Ω to 100Ω. The impedance Z1 is set to 30Ω if a tissue to be treated is a blood vessel and is set to 50Ω if the tissue to be treated is a parenchyma organ.

### <Step S14>

If the impedance Z becomes not less than the impedance Z1 that is the first predetermined value (YES in S13), the control section 94 acquires a low-impedance time period t1 (see FIG. 6) which is a duration time period from when the HF energy application starts to when the impedance Z becomes not less than the impedance Z1.

Note that a time period from when the impedance becomes the minimum impedance Zmin to when the impedance becomes not less than the impedance Z1 may be used as the low-impedance time period.

### <Step S15>

The control section 94 calculates an end impedance Zf on the basis of the low-impedance time period t1. The end impedance Zf is a threshold value, based on which completion of the process of destroying cell membranes in the grasped living tissue is sensed, and application of high-frequency power energy is ended.

In the treatment system 10, table A illustrated in (Table 1) below, which is based on experimental data and is data for acquiring the end impedance Zf on the basis of the low-impedance time period t1, is stored in advance in the memory 92.

TABLE 1
Table A

| T1 (ms) | Zf (Ω) |
|---|---|
| 1500 | 450 |
| 2000 | 500 |
| 3000 | 500 |
| 4000 | 600 |
| 5000 | 600 |
| 6000 | 700 |
| 7000 | 700 |
| 7500 | 750 |

Note that the control section 94 may calculate the end impedance Zf on the basis of the calculation formula Zf=f(t1). In the case of the power supply portion 12, to which a plurality of different treatment instruments can be connected, a plurality of tables corresponding to the respective treatment instruments can be stored in the memory 92 such that one of the tables can be selected according to the connected treatment instrument.

### <Step S16>

When drying of the living tissue advances further by further application of HF energy, the impedance Z rises further. At the time of HF energy application, the high impedance Z not only makes appropriate energy application difficult but also makes arc discharge likely to occur.

The HF control section 74 determines whether the impedance Z is not less than the end impedance Zf. If the HF control section 74 determines that the impedance Z is less than the end impedance Zf (NO in S16), the HF control section 74 continues applying HF energy.

### <Step S17>

On the other hand, if the HF control section 74 determines that the impedance Z is not less than the end impedance Zf (YES in S16), the HF control section 74 controls the high-frequency power source 73 to stop outputting HF.

If the HF control section 74 determines that the impedance Z has become not less than the end impedance Zf, a signal is transmitted from the HF control section 74 of the HF unit 72 to the TH control section 84 of the TH unit 82 via the communication line 91. Switching from HF energy application mode to TH energy application mode is performed.

### <Step S18>

In TH energy application mode, the TH control section 84 supplies TH to the heater member 53 such that the temperature of the heater member 53 is the predetermined setting temperature Tset (e.g., about 120° C. to 300°). That is, the TH control section 84 performs feedback control that increases/decreases TH output on the basis of a temperature T of the heater member 53.

The treatment in HF energy application mode has made the living tissue uniformed and has raised thermal conductivity. For the reason, in TH energy application mode, heat from the heater member 53 is efficiently transferred to the living tissue. In TH energy application mode, proteins in the living tissue are integrally denatured, and removal of moisture that is a hindrance to hydrogen bonding between proteins is performed, in order to join the living tissue through hydrogen bonding.

Hydrogen bonding is a noncovalent attractive interaction which a hydrogen atom covalently bonded to a highly electronegative atom (an electronegative atom) forms with a lone pair of electrons such as on nitrogen, oxygen, sulfur, or fluorine or in a π-electron system that is located in a neighborhood. In proteins in a living tissue, a hydrogen bond is formed between an oxygen atom in a main chain and a hydrogen atom of an amide bond. Unlike simple joining through denaturation of proteins, moisture content control and temperature control at the time of joining are important for joining through hydrogen bonding. For the controls, precise control of thermal energy to be applied is important.

As shown in FIG. 7, the temperature T of the heater member 53 at a TH energy application mode start time (t=0), i.e., an HF energy application mode end time (t=tf) is, for example, 100° C. Application of TH energy that is brought under constant temperature control aiming for the predetermined setting temperature Tset causes the temperature T of the heater member 53 to rise to the setting temperature Tset (e.g., 180° C.) and then be held at the setting temperature Tset. That is, the setting temperature Tse is set to be higher than the temperature T of the heater member 53 at the HF energy application mode end time (t=tf).

Power (TH) for heat generation is high until the temperature rises to the setting temperature Tset. In other words, since temperature of the grasped living tissue having high thermal capacity needs to be raised in order to raise the temperature T of the heater member 53, the TH needs to be high.

Note that the TH exhibits a fixed value (THmax) from a time t1 to a time t2 in FIG. 7 because the value THmax is set to a maximum rated power (e.g., 100 W) of the power source 83 for heat generation. The value THmax is set to the maximum rated power because a power source having high maximum rated power is expensive and large. Note that the treatment system 10 does not matter much even when an inexpensive power source having low maximum rated power is used.

After the temperature T of the heater member 53 reaches the setting temperature Tset, the TH required to maintain the temperature Tse becomes low. As the treatment advances further, and contraction or the like of the grasped living tissue advances, the TH becomes lower.

### <Steps S19 and S20>

The TH control section 84 determines whether the TH is not more than the predetermined end power THf. The end power THf set in step S10 is, for example, 10 W to 30 W.

If the TH control section 84 determines that the TH has exceeded the predetermined end power THf (NO in S19), the TH control section 84 continues applying TH energy. On the other hand, if the TH control section 84 determines that the TH is not more than the predetermined end power THf (YES in S19), the TH control section 84 ends application of TH energy in step S20 and completes the treatment (t9 in FIG. 7).

As described above, the treatment system 10 ends application of high-frequency power energy on the basis of the end impedance Zf that is a threshold value calculated based on the low-impedance time period t1 that is a time period for the impedance Z for high-frequency power to become not less than the first predetermined value (the impedance Z1).

That is, although the minimum impedance Zmin is of some benefit in determining when to end application of high-frequency power energy, the application cannot be ended at an appropriate time only on the basis of the minimum impedance Zmin in some cases. For the reason, a conventional treatment system may not be said to have good operability.

The low-impedance time period t1 more accurately reflects change in status of the living tissue being treated than the minimum impedance Zmin.

The treatment system 10 and the actuation method for the treatment system 10 are capable of switching from application of high-frequency power energy to application of thermal energy at an appropriate time and are thus good in operability.

Note that the high-frequency power source 73 and the power source 83 for heat generation do not simultaneously output power in the treatment system 10. For the reason, one common power source may function as a high-frequency power source or a power source for heat generation under control of the control section 94.

In a treatment system in which respective heater members are disposed at the jaws 36a and 36b of the treatment instrument 11, respective power sources for heat generation may be controlled on the basis of temperatures of the heater members. Alternatively, control may be performed by one power source for heat generation on the basis of an average temperature of the two heater members.

### Modification of First Embodiment

A basic configuration of the treatment system 10A according to the modification of the first embodiment is substantially identical to a basic configuration of the treatment system 10. A control section 94A of the treatment system 10A calculates, as a threshold value at which application of high-frequency power energy is ended, a rate ZVf of change in end impedance which is a rate of change in impedance Z on the basis of a low-impedance time period t1.

Like an end impedance Zf, the rate ZVf of change in end impedance is stored in advance as a table illustrated in (Table 2) which is data calculated on the basis of experimental data in the memory 92.

TABLE 2

| T1 (ms) | ZVf (Ω/ms) |
|---|---|
| 1500 | 0.06 |
| 2000 | 0.05 |
| 3000 | 0.05 |
| 4000 | 0.04 |
| 5000 | 0.04 |
| 6000 | 0.03 |
| 7000 | 0.03 |
| 7500 | 0.03 |

Note that the control section 94A may calculate the rate ZVf of change in end impedance that is a threshold value on the basis of the calculation formula ZVf=f(t1).

The rate ZVf of change in end impedance that is calculated on the basis of the low-impedance time period t1 can be used as a reference (threshold value) for switching from application of high-frequency power energy to application of thermal energy at an appropriate time, like the end impedance Zf.

That is, the treatment system 10A and an actuation method for the treatment system 10A have the same effects as effects of the treatment system 10 or the like.

### Second Embodiment

A treatment system 10B according to a second embodiment will be described. Since the treatment system 10B is similar to the treatment system 10 or the like, constituent elements having identical functions are denoted by identical reference numerals, and a description of the constituent elements will be omitted.

In the treatment system 10 according to the first embodiment, the impedance Z1 that is the first predetermined value is set by a surgeon according to the type of a living tissue to be treated at the start of treatment. In contrast, in the treatment system 10B, the type of a living tissue is automatically determined after treatment starts, and an impedance Z1 is automatically set. That is, in the treatment system 10B, a control section 94B calculates an end impedance Zf that is a threshold value at which application of high-frequency power energy is ended, on the basis of a minimum impedance Zmin and a low-impedance time period t1.

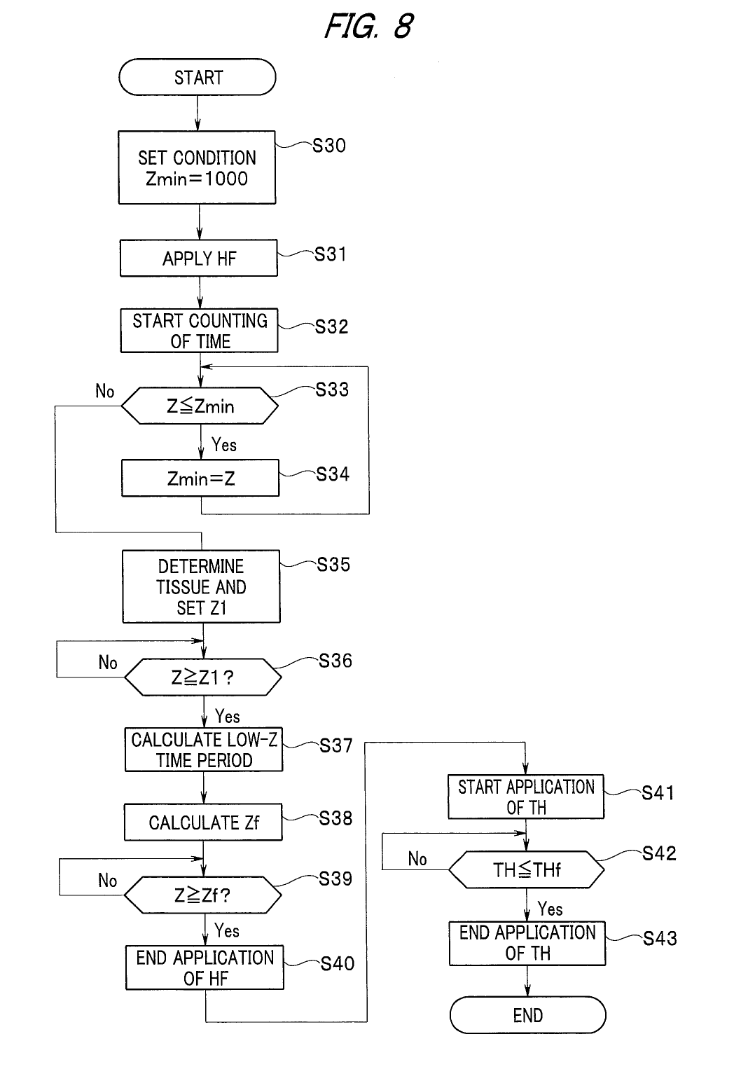

A motion of the treatment system 10B will be described below with reference to a flowchart shown in FIG. 8.

### <Step S30>

Step S30 is substantially same as S10 of the flow chart shown in FIG. 5. Note that a sufficiently large value (e.g., 1000Ω) is substituted as an initial value into the minimum impedance Zmin in the treatment system 10B. The impedance Z1 that is a first predetermined value is not set.

### <Steps S31 and S32>

Steps S31 and S32 are substantially same as S11 and S12 of the flow chart shown in FIG. 5.

### <Steps S33 and S34>

If an impedance Z is not more than the minimum impedance Zmin (YES in S33), the impedance Z is substituted into the minimum impedance Zmin in step S34. With repetition of the process, the minimum impedance Zmin is acquired.

### <Step S35>

The control section 94B automatically determines the type of a living tissue being treated on the basis of the minimum impedance Zmin.

A description below will be given in the context of a case where a living tissue is determined as one of two types, for simplicity of explanation.

In the treatment system 10B, data for determining the type of a grasped living tissue on the basis of the minimum impedance Zmin is stored in advance as table B illustrated in (Table 3) in a memory 92.

TABLE 3
Table B

| Zmin (Ω) | Tissue | Table A |
|---|---|---|
| 13.0 | Bronchus | A1 |
| 13.5 | | |
| 14.0 | | |
| 14.5 | | |
| 15.0 | | |
| 15.5 | Lungs (Edge) | A2 |
| 16.0 | | |
| 16.5 | | |
| 17.0 | | |
| 17.5 | Lungs (Center) | |
| 18.0 | | |
| 18.5 | | |
| 19.0 | | |

The control section 94B determines the type of the grasped living tissue on the basis of the minimum impedance Zmin by using table B illustrated in (Table 3). The impedance Z1 is automatically set according to the type of the living tissue.

As for table B illustrated, the grasped living tissue is determined as a “bronchus” if the minimum impedance Zmin is not more than 15.25Ω. The grasped living tissue is determined as a “lung” if the minimum impedance Zmin is more than 15.25Ω. The control section 94B may, of course, simply determine the type as, e.g., “type A1” or “type A2” instead of specifically determining the type of a living tissue on the basis of the minimum impedance Zmin.

### <Steps S36 and S37>

If the impedance Z becomes not less than the impedance Z1 that is the first predetermined value (YES in S36), the control section 94B acquires the low-impedance time period t1 that is a duration time period from when the application of HF energy starts to when the impedance Z becomes not less than the impedance Z1.

Note that a time period from when the impedance Z reaches the minimum impedance Zmin to when the impedance Z becomes not less than impedance Z1 may be used as the low-impedance time period.

### <Step S38>

The control section 94B calculates the end impedance Zf that is a threshold value on the basis of the low-impedance time period t1.

That is, in the treatment system 10B, table A1 illustrated in (Table 4) and table A2 illustrated in (Table 5) below that are based on experimental data and are data for acquiring the end impedance Zf on the basis of the low-impedance time period t1 are stored in advance in the memory 92.

TABLE 4
Table A1

| T1 (ms) | Zf (Ω) |
|---|---|
| 1500 | 450 |
| 2000 | 500 |
| 3000 | 500 |
| 4000 | 600 |
| 5000 | 600 |
| 6000 | 700 |
| 7000 | 700 |
| 7500 | 750 |

TABLE 5
Table A2

| T1 (ms) | Zf (Ω) |
|---|---|
| 2000 | 750 |
| 3000 | 700 |
| 4000 | 650 |
| 5000 | 600 |
| 6000 | 550 |

The control section 94B uses a table corresponding to the type of the living tissue determined in step S35.

If the tissue is determined as a “bronchus (type A1)”, table A1 is selected. If the tissue is determined as a “lung (type A2)”, table A2 is selected.

The control section 94B may, of course, select table A1 or A2 directly on the basis of the minimum impedance Zmin.

The control section 94B calculates the end impedance Zf from the low-impedance time period t1 by using selected table A1 or A2.

For example, if table A1 is selected, and the low-impedance time period t1 is 3000 ms, the end impedance Zf is 500Ω. On the other hand, if table A2 is selected, the end impedance Zf is 700Ω even when the low-impedance time period t1 is identical to 3000 ms.

Note that the control section 94 may calculate the end impedance Zf on the basis of the minimum impedance Zmin by using a calculation formula selected from among a plurality of calculation formulae.

### <Steps S39 to S43>

Steps S39 to S43 are substantially same as S16 to S20 of the flow chart shown in FIG. 5.

The treatment system 10B and an actuation method for the treatment system 10B have the same effects as effects of the treatment system 10 or the like. Additionally, the treatment system 10B can end application of HF energy at an appropriate time even if a living tissue to be treated switches to a different one.

Note that a rate ZVf of change in end impedance that is a rate of change in impedance Z may also be calculated, as a threshold value at which application of high-frequency power energy is ended, on the basis of the low-impedance time period t1 in the treatment system 10B, as in the treatment system 10A.

Like the treatment system 10, one common power source may function as a high-frequency power source or a power source for heat generation under control of the control section 94B.

### Third Embodiment

A treatment system 10C according to a third embodiment will be described. Since the treatment system 10C is similar to the treatment system 10 or the like, constituent elements having identical functions are denoted by identical reference numerals, and a description of the constituent elements will be omitted.

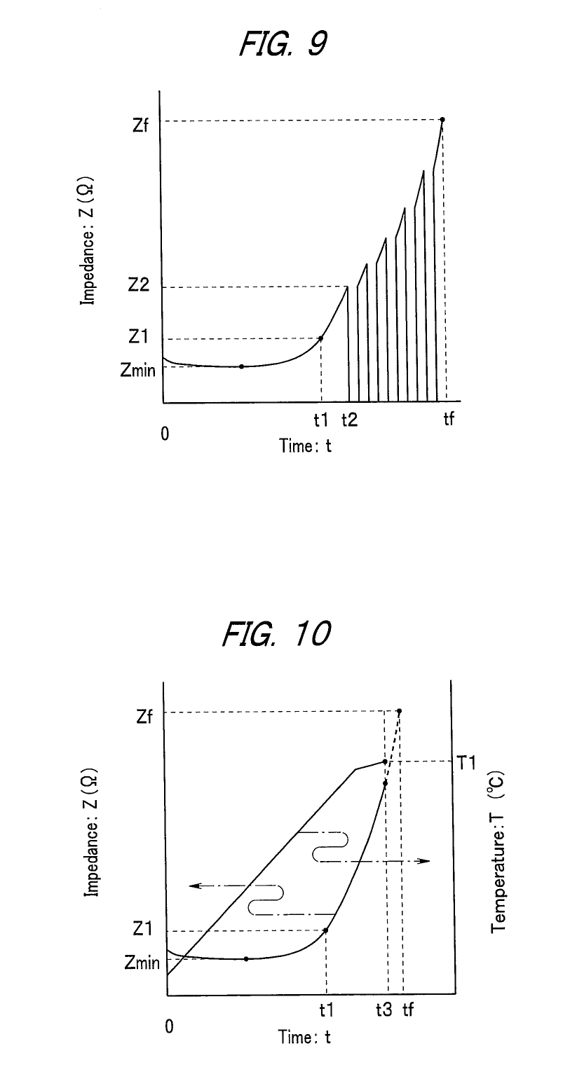

As shown in FIG. 9, a control section 94C of the treatment system 10C controls a high-frequency power source 73 so as to change HF output from previous continuous output to intermittent output (pulsed output) when impedance for high-frequency power becomes not less than a second predetermined value Z2. The second predetermined value Z2 may be stored in advance in a memory 92 or the like, may be set by a surgeon, or may be calculated from an impedance Z1, an end impedance Zf, or the like by the control section 94C.

When application of HF energy advances drying of a living tissue, a rate of rise in impedance Z increases. For the reason, it may not be easy to end HF energy application mode at an appropriate time.

However, when the impedance Z becomes not less than the predetermined value Z2 in the treatment system 10C (t2 in FIG. 9), HF is outputted as pulsed output, and the amount of energy applied per unit time decreases. For example, if a duty ratio (ON time/(ON time+OFF time)) of the pulsed output is 0.5, the amount of energy applied per unit time is half. The rate of rise in impedance Z is thus substantially half.

For the reason, the treatment system 10C and an actuation method for the treatment system 10C have the same effects as effects of the treatment system 10 or the like. Additionally, the treatment system 10C more easily can end HF energy application mode at a more appropriate time.

### Fourth Embodiment

A treatment system 10D according to a fourth embodiment will be described. Since the treatment system 10D is similar to the treatment system 10 or the like, constituent elements having identical functions are denoted by identical reference numerals, and a description of the constituent elements will be omitted.

In the treatment system 10 or the like already described, switching from HF energy application mode to TH energy application mode is automatically performed. The mode switching, however, may be late, depending on, e.g., a grasping status of a living tissue.

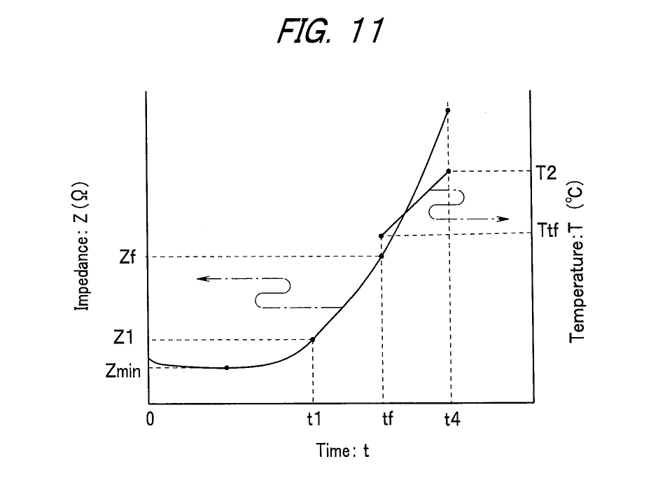

As shown in FIG. 10, in the treatment system 10D, a control section 94D calculates temperature of a heater member 53 by applying power for heat generation for monitoring to a heater member 53 which is a heating element during application of HF energy. If the temperature of the heater member 53 is not less than a predetermined first temperature T1 (t3 in FIG. 10), the control section 94D ends application of HF energy even when an impedance Z for HV is less than an end impedance Zf.

The TH for monitoring here refers to power for measuring the temperature which is lower than TH for applying thermal energy. For example, the TH for applying thermal energy is about 20 W to 150 W, and the TH for monitoring is about 1 W to 5 W. For the reason, the heater member 53 produces little thermal energy even if the power for heat generation for monitoring is applied.

The first temperature T1, based on which application of HF energy ends, is preferably, for example, not less than 100° C. and less than 130° C. in order to prevent a living tissue being treated from being excessively cauterized through application of excessive HF energy.

The treatment system 10D and an actuation method for the treatment system 10D have effects of the treatment system 10 or the like. Additionally, HF energy application mode can be appropriately ended before excessive cauterization occurs even in a case where HF energy application mode ends late on the basis of the end impedance Zf calculated based on a low-impedance time period t1.

Note that since the high impedance Z makes appropriate energy application difficult, a temperature T of the heater member 53 starts decreasing after exhibiting a maximum temperature Tmax. For the reason, the control section 94D may end application of HF energy if the temperature T of the heater member 53 declines by a predetermined temperature ΔT from the maximum temperature Tmax. The predetermined temperature ΔT is preferably, for example, not less than 5° C. and less than 30° C.

That is, in the treatment system 10D, the control section 94D calculates, from a resistance value of the heater member 53, the temperature of the heater member 53 that exhibits the maximum temperature Tmax and then decreases due to application of HF energy. If the temperature of the heater member 53 is not less than the first temperature T1 or if the temperature of the heater member 53 decreases by the predetermined temperature ΔT or more from the maximum temperature Tmax, the control section 94D ends application of HF energy.

### Fifth Embodiment

A treatment system 10E according to a fifth embodiment will be described. Since the treatment system 10E is similar to the treatment system 10 or the like, constituent elements having identical functions are denoted by identical reference numerals, and a description of the constituent elements will be omitted.

In the treatment system 10 or the like already described, switching from HF energy application mode to TH energy application mode is automatically performed. The mode switching, however, may be early, depending on, e.g., a grasping status of a living tissue.

As shown in FIG. 11, in the treatment system 10E, a control section 94E calculates a temperature T of a heater member 53 which is a heating element when the control section 94E ends application of HF energy on the basis of an end impedance Zf which is calculated based on a low-impedance time period t1. If the temperature T of the heater member 53 is not more than a predetermined second temperature T2, the control section 94E does not start application of thermal energy and restarts applying HV energy. The second temperature T2 here is preferably, for example, not less than 100° C. and less than 110° C. in order to reliably complete a process of destroying cell membranes in a living tissue being treated.

That is, the control section 94E ends HF energy application mode when an impedance Z becomes not less than the end impedance Zf, and the temperature of the heater member 53 becomes not less than the second temperature T2.

The treatment system 10E and an actuation method for the treatment system 10E have effects of the treatment system 10 or the like. Additionally, HF energy application mode can be appropriately ended after the process of destroying cell membranes is completed even in a case where HF energy application mode ends early on the basis of the end impedance Zf calculated based on the low-impedance time period t1.

Note that although FIG. 11 illustrates a case where the temperature T of the heater member 53 is not measured when the impedance Z is less than the end impedance Zf, measurement of the temperature T may be started at a same time as the start of application of HV energy, as in the treatment system 10D.

Note that two or more of the configurations of the embodiments and the modification described above may be used in combination. For example, the configuration of the treatment system 10C, the configuration of the treatment system 10D, and the configuration of the treatment system 10E can be used in combination.

As described above, a treatment system according to the embodiment includes a high-frequency power source which outputs high-frequency power, a power source for heat generation which outputs power for heat generation, one pair of grasping members having an electric conductor which applies the high-frequency power as high-frequency power energy to a grasped living tissue and is disposed at each of the two grasping members and a heating element which applies the power for heat generation as thermal energy to the living tissue, which is disposed at at least one of the grasping members, and which is made of a material having a positive temperature coefficient of resistance, a memory where data for acquiring a threshold value, at which application of the high-frequency power energy is ended, on the basis of a low-impedance time period is stored in advance, the low-impedance time period being a time period for an impedance for the high-frequency power which decreases after application of the high-frequency energy under constant power control starts and increases after exhibiting a minimum value to become not less than a first predetermined value, a first control section which ends application of the high-frequency power energy on the basis of the threshold value acquired based on the low-impedance time period of the impedance for the high-frequency power that is detected after application of the high-frequency energy starts by using the data stored in the memory, and a second control section which automatically performs constant temperature control on the power source for heat generation such that the heating element reaches a predetermined temperature which is higher than a temperature calculated from resistance of the heating element after application of the high-frequency power energy ends.

A control method for a treatment system according to the embodiment includes a step of setting a treatment condition for a treatment system including a high-frequency power source which outputs high-frequency power, a power source for heat generation which outputs power for heat generation, and one pair of grasping members having an electric conductor which applies the high-frequency power as high-frequency power energy to a grasped living tissue and is disposed at each of the two grasping members and a heating element which applies the power for heat generation as thermal energy to the living tissue, which is disposed at at least one of the grasping members, and which is made of a material having a positive temperature coefficient of resistance, a step of starting application of the high-frequency energy and acquiring a low-impedance time period, the low-impedance time period being a time period for an impedance for the high-frequency power which decreases after application of the high-frequency energy starts and increases after exhibiting a minimum value under constant power control based on the treatment condition to become not less than a first predetermined value, a step of acquiring a threshold value, at which application of the high-frequency power energy is ended, from the minimum value and the low-impedance time period on the basis of data stored in a memory, a step of automatically ending application of the high-frequency energy on the basis of the threshold value, a step of applying the thermal energy under constant temperature control on the power source for heat generation such that the heating element reaches a predetermined temperature which is higher than a temperature calculated from resistance of the heating element when application of the high-frequency energy is ended, and a step of ending application of the thermal energy on the basis of the treatment condition.

The present invention is not limited to the above-described embodiments and the like. Various changes, modifications, and the like can be made without departing from the scope of the present invention.

What is claimed is:

1. A treatment system comprising:
a high-frequency power source which outputs high-frequency power;
a power source for heat generation which outputs power for heat generation;
a grasping member having an electric conductor which applies the high-frequency power as high-frequency power energy to a living tissue and a heating element which applies the power for heat generation as thermal energy to the living tissue;
a memory where a predetermined impedance value or a predetermined rate of increase in impedance that is a threshold value, at which application of the high-frequency power energy is ended, is stored in advance;
a first control section which controls the high-frequency power source on the basis of the threshold value so as to end application of the high-frequency power energy; and
a second control section which performs constant temperature control on the power source for heat generation such that the heating element reaches a setting temperature that is set to be higher than a temperature of the heating element calculated from a resistance value of the heating element when application of the high-frequency power energy ends.

2. The treatment system according to claim 1, wherein
the threshold value stored in the memory is based on a low-impedance time period which is a time period for an impedance for the high-frequency power to become not less than a first predetermined value during application of the high-frequency energy.

3. The treatment system according to claim 2, wherein
data for determining a type of the living tissue on the basis of a minimum value for the impedance for the high-frequency power is stored in advance in the memory, and
the first control section acquires the first predetermined value from the minimum value for the impedance for the high-frequency power and the data stored in the memory and automatically sets the first predetermined value.

4. The treatment system according to claim 2, wherein
a process of destroying a cell membrane in the living tissue is completed through application of the high-frequency power energy, moisture is removed from the living tissue through application of the thermal energy, and the living tissue is joined through hydrogen bonding.

5. The treatment system according to claim 2, wherein
the first control section controls the high-frequency power source so as to change the high-frequency power from continuous output to intermittent output when the impedance for the high-frequency power becomes not less than a second predetermined value.

6. The treatment system according to claim 2, wherein
the high-frequency power source and the power source for heat generation are made up of a common power source.

7. The treatment system according to claim 2, wherein
the temperature of the heating element is calculated during application of the high-frequency power energy, and the first control section controls the high-frequency power source so as to end application of the high-frequency power energy if the temperature of the heating element is not less than a first temperature.

8. The treatment system according to claim 2, wherein
the temperature of the heating element is calculated when application of the high-frequency power energy is ended, and the second control section controls the power source for heat generation so as not to start application of the thermal energy but to restart application of the high-frequency power energy if the temperature of the heating element is not more than a predetermined second temperature.

9. An actuation method for a treatment system, comprising:
a step in which a treatment condition is set for the treatment system including a first control section which controls the high-frequency power source for applying high-frequency power as high-frequency power energy to an electric conductor and a second control section which controls the power source for heat generation for applying power for heat generation as thermal energy to a heating element;
a step of performing, by the first control section, constant power control on the high-frequency power source on the basis of the treatment condition to start application of the high-frequency energy;
a step of acquiring, by the first control section, a predetermined impedance value or a predetermined rate of increase in impedance that is a threshold value, at which application of the high-frequency power energy is ended, from a memory;
a step of controlling, by the first control section, the high-frequency power source to automatically end application of the high-frequency energy on the basis of the threshold value;
a step of applying, by the second control section, the thermal energy under constant temperature control on the power source for heat generation such that the heating element reaches a setting temperature that is set to be higher than a temperature of the heating element when application of the high-frequency energy is ended which is calculated from resistance of the heating element; and
a step of controlling, by the second control section, the power source for heat generation on the basis of the treatment condition so as to end application of the thermal energy.

10. The actuation method for the treatment system according to claim 9, wherein
during application of the high-frequency energy,
the first control section acquires a low-impedance time period which is a time period for an impedance for the high-frequency power to become not less than a first predetermined value, and
the first control section acquires the threshold value from the memory on the basis of the low-impedance time period.

11. The actuation method for the treatment system according to claim 10, wherein
before the low-impedance time period is acquired, the first control section acquires the first predetermined value from a minimum value for the impedance for the high-frequency power on the basis of data for determining a type of the living tissue stored in the memory and automatically acquires the first predetermined value.

12. The actuation method for the treatment system according to claim 9, wherein
a process of destroying a cell membrane in the living tissue is completed through application of the high-frequency power energy by the first control section, moisture is removed from the living tissue through application of the thermal energy by the second control section, and the living tissue is joined through hydrogen bonding.

13. The actuation method for the treatment system according to claim 9, wherein
during application of the high-frequency power energy, the high-frequency power source is controlled by the first control section so as to change the high-frequency power from continuous output to intermittent output when the impedance for the high-frequency power becomes not less than a second predetermined value.

14. The actuation method for the treatment system according to claim 9, wherein
the high-frequency power source and the power source for heat generation are made up of a common power source.

15. The actuation method for the treatment system according to claim 9, wherein
the temperature of the heating element is calculated during application of the high-frequency power energy, and the high-frequency power source is controlled by the first control section to end application of the high-frequency power energy if the temperature of the heating element is not less than a first temperature.

16. The actuation method for the treatment system according to claim 9, wherein
the temperature of the heating element is calculated when application of the high-frequency power energy is ended, and if the temperature of the heating element is not more than a second temperature, application of the thermal energy is not started, and the high-frequency power source is controlled by the first control section to restart application of the high-frequency power energy.

17. A treatment system comprising:
a high-frequency power source which outputs high-frequency power;
a power source for heat generation which outputs power for heat generation;
one pair of grasping members which grasp a living tissue, the grasping members having an electric conductor which applies the high-frequency power as high-frequency power energy and is disposed at each of the two grasping members and a heating element which applies the power for heat generation as thermal energy to the living tissue, which is disposed at at least one of the grasping members, and which is made of a material having a positive temperature coefficient of resistance;
a memory where data for acquiring a threshold value, at which application of the high-frequency power energy is ended, on the basis of a low-impedance time period is stored in advance, the low-impedance time period being a time period for an impedance for the high-frequency power which decreases after application of the high-frequency energy under constant power control starts and increases after exhibiting a minimum value to become not less than a first predetermined value;
a first control section which ends application of the high-frequency power energy on the basis of a threshold value that is acquired based on the low-impedance time period of the impedance for the high-frequency power detected after application of the high-frequency energy starts by using the data stored in the memory; and
a second control section which automatically performs constant temperature control on the power source for heat generation such that the heating element reaches a predetermined temperature which is higher than a temperature calculated from resistance of the heating element after application of the high-frequency power energy ends.
